(19) United States
(12) Patent Application Publication
(10) Pub. No.: US 2003/0194719 A1
(43) Pub. Date: Oct. 16, 2003

(54) Tpst-assay for diagnosis of autism and related disorders

(76) Inventors: Rosemary Waring, Birmingham (GB); Joanne Phoenix, Wirral (GB)

Correspondence Address: Thomas Q Henry, Woodard Emhardt Naughton Moriaty & McNett, Bank One Tower Suite 3700, 111 Monument Circle, Indianapolis, IN, 46204, US

(21) Appl. No.: 10/240,863

(22) PCT Filed: Apr. 5, 2001
(86) PCT No.: PCT/GB01/01569

(30) Foreign Application Priority Data
Apr. 6, 2000 (GB) 0008326.1

(51) Int. Cl. C12Q 1/68; G01N 33/53; G01N 33/537; G01N 33/543

(52) U.S. Cl. 435/6; 435/7.92

(57) ABSTRACT

The present invention relates to a method for diagnosing or detecting a predisposition to autism and related diseases comprising assaying a bodily sample in vitro directly or indirectly for a reduced tyrosyl protein sulphotransferase (TPST) level as compared to a reference sample, and a method of treatment for the same diseases by administering to a patient suffering therefrom a therapeutic amount of an enhancer of TPST.

The present invention relates to the diagnosis and treatment of autism and related disorders such as those involving language problems and obsessive compulsive traits, disease associated with food allergy, diseases commonly treated with dopamine antagonists and diseases which have an associated abnormal cholecystokinin activity.

Autism is a pervasive developmental disorder generally characterised by withdrawal from social contact and repetitive or obsessive behavior, often with bursts of hyperactivity. Autism is characterised by a broad phenotype and attempts to define a genotype have been hindered by this fact. However, recent approaches to define sub-populations within the phenotype using personality characteristics of families hold promise for revealing associated genes. Using this strategy, associations with regions on chromosomes 7, 13 and 15 have been found which can be linked to pro-bands showing language problems (chromosomes 7 and 13) and obsessive compulsive traits (chromosome 15). High rates of duplication in 15q 11-13 are also emerging, and this region is also implicated in Angelman's syndrome. It has also been demonstrated that an over-expression of the D8/17 immune marker is particularly associated with pro-bands demonstrating obsessive compulsive traits, and this marker is also implicated in the incidence of rheumatic fever, Sydenham's Chorea, childhood-onset obsessive compulsive disorder, Tourettes syndrome and the development of tics.

Neuro-functional abnormalities have been suggested in autism and many theories extended as explanations for the diverse symptoms of the condition. Oxytocin and vasopressin have been implicated in the neurophysiological network that regulates social affiliation and imprinting, and evidence has been collected which demonstrates an oxytocin deficiency in autism.

One hypothesis to explain the diverse aspects of autism is the opioid excess theory, initially proposed by Panksepp (1979). The theory suggests that excessive absorption of dietary peptides leads to disruption of biochemical and neuroregulatory processes resulting in the diverse symptoms which manifest as autism. The peptides thought to pose the biggest problem are those which are formed from the incomplete or abnormal digestion of gluten and casein producing peptides having opioid activity.

B-casomorphins in particular have been shown to function as exogenous opioids and have profound effects in rats. The levels of these peptides decrease on removal of gluten and/or casein from the diet, coinciding with an improvement in autistic behavior. Benefits of a gluten-free and/or casein-free diet include increased levels of attention and concentration; decreased aggression; improved sleep, communication, physical well-being and eating habits, as reported by parents and teachers and confirmed by the psychological and educational test procedures employed by researchers. Gluten and casein sensitivity are now considered to be, at least in part, primary to the aetiology of autism and are genetically determined.

Secretin is a hormone involved in stimulating digestion. The benefits of secretin for the treatment of autism have been related to the promotion of adequate digestion of the offending peptides in the diet, but it remains to be seen whether this remains a viable treatment option in the light of a negative result in a properly controlled trial. Research activities have also suggested that the incidence of autism can be linked to a defect in dipeptidyl-peptidase IV enzyme in the gut, which breaks peptides down into amino acids however it remains to be seen whether modulation of the dipeptidyl-peptidase IV enzyme would provide an effective treatment for autism.

Tyrosyl protein sulphotransferase (T?ST) catalyses the transfer of sulphate from 3′-phosphoadenosine 5′ phosphosulphate (PAPS) to tyrosine residues within highly acidic regions of polypeptides. Tyrosine sulphation is a widespread post-translational modification of proteins and peptides. The majority of sulphated proteins and peptides are secretory and range from small peptide hormones, such as CCK and gastrin, to much larger proteins like immunoglobulins, coagulation factors and proteins of the extracellular matrix which promote cell-substratum or cell-cell adhesion and provide guidance for neuronal morphogenesis and migration.

The main effects of TPST within the digestive system are shown below:

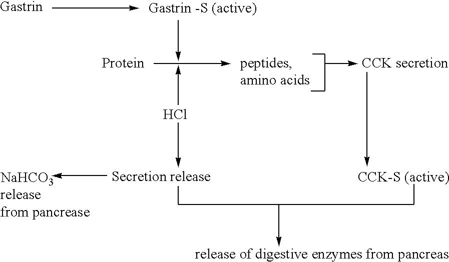

TPST is a membrane-associated protein with a luminally-orientated active site localised in the trans-Golgi network. cDNA probes have been made and the complete amino acid sequence determined (Ouyang et al., 1998), revealing homology to a large family of cytosolic sulphotransferases, including phenol and hydroxysteroid sulphotransferases.

It has previously been reported that plasma sulphate levels and levels of phenolsulphotransferase activity are reduced compared to normal in autistic individuals (O'Reilly & Waring, 1993; Waring et al., 1997) leading to the build up of phenols which could be implicated in disturbed metabolic processes.

Abnormal TPST activity in the nervous system is of particular interest because regions demonstrating high levels of TPST activity in the rat brain normally produce vasopressin and oxytocin, which have previously been implicated in the neurophysiological network that regulates social affiliation and imprinting.

Several neuropeptides are modulated by tyrosine sulphation by TPST. For example TPST acts to sulphate, and therefore activate, CCK in the brain. CCK in the brain is widespread and the predominant form is CCK-8, an octapeptide whose sole tyrosine residue is sulphated by TPST. CCK-immunoreactive neurons are present in significant numbers in the hippocampus, amygdala, claustrum, bed nucleus of the stria terminalis and a number of thalamic and hypothalamic nuclei. CCK coexists with dopamine and neurotensin in the substantia nigra and ventrotegmental area, with vasoactive intestinal peptide (VIP), neuropeptide Y (NPY) and γ-aminobutyric acid (GABA) in the thalamocortical and thalamostriatal connections, and with substance P and serotonin (5-HT) in medullary neurons. It is present in the dorsal root ganglia and coexists with pro-enkephalin-derived peptides (which have opioid activity) in both the cortex and thalamus.

TPST activity may be modulated. Ethanol has been shown to increase TPST level by over three-fold in the stomach and liver of ethanol-fed male Sprague-Dawley rats and the same has also been shown to be true in human alcoholics, with enzyme activity two- to three-fold higher than in normal individuals. Manganese suppresses TPST activity in PC12 cells possibly via interference in triacyglycerol metabolism and subsequent alteration in the lipid composition of the Golgi apparatus. TPST is proposed to be a lipid-dependent enzyme and physiological concentrations of prostaglandins stimulate activity of TPST in a dose-dependent manner.

It is an aim of a preferred embodiment of the present invention to obviate or mitigate any problem associated with the prior art and to provide a diagnostic test for autism and related disorders and to provide for methods and compositions for treatment of the same.

According to the present invention there is provided a method for diagnosing or detecting a predisposition to autism and related diseases comprising assaying a bodily sample in vitro directly or indirectly for a reduced tyrosyl protein sulphotransferase level as compared to a reference sample.

The invention is based on the inventors' finding that TPST levels are significantly reduced in platelet samples from autistic patients as compared to healthy patients.

Whilst not wishing to be bound by theory the inventors have the following proposals as to why a decreased level of TPST is associated with autism and related diseases.

The first prong of the inventors' proposal is based on the activation of neuropeptides in the brain by TPST. Several neuropeptides are known to be modulated by tyrosine sulphation and this occurs in many proteins and peptides important to the development and functional state of the nervous system. The action of TPST to sulphate, and therefore activate, CCK in the brain deserves further consideration here

CCK coexists with dopamine and neurotensin in the substantia nigra and ventrotegmental area, with vasoactive intestinal peptide (VIP), neuropeptide Y (NPY) and γ-aminobutyric acid (GABA) in the thalamocortical and thalamostriatal connections, and with substance P and serotonin (5-HT) in medullary neurons. CCK is present in the dorsal root ganglia and coexists with pro-enkephalin-derived peptides (which have opioid activity) in both the cortex and thalamus. Exogenous CCK has opioid antagonistic properties. For all these reasons the inventors propose that CCK may act as a control mechanism for these, and a number of different neuropeptides.

As expected from its widespread distribution in the brain, CCK has been implicated in a number of physiological and behavioral processes, including neuroendocrine regulation, pain transmission, learning and memory and exploratory behavior. It has been proposed that interference with normal CCK function may disrupt tissue growth and normal mammalian development and it has been suggested that CCK may play an important role in the maturation of the cortical circuitry that mediates the acquisition of certain cognitive abilities. The inventors therefore propose that a failure of adequate activation of CCK by TPST would have widespread implications on a number of aspects which have been associated with certain conditions such as autism, including control of emotional and sensory tone (e.g. amygdala involvement).

CCK may function as an excitatory neurotransmitter and can have anxiolytic effects. That CCK can also interact with dopamine in an inhibitory manner reinforces the inventors' proposal that CCK acts as a control mechanism for neuropeptides and that TPST may modulate this control mechanism.

Neurofunctional abnormalities have been suggested in autism and many theories extended as explanations for the diverse symptoms of the condition. Oxytocin and vasopressin have been implicated in the neurophysiological network that regulates social affiliation and imprinting, and evidence has been collected which demonstrates an oxytocin deficiency in autism. The inventors propose that abnormal TPST activity in the nervous system is of particular interest because regions demonstrating high levels of TPST activity in the rat brain normally produce vasopressin and oxytocin. The inventors therefore propose that a primary TPST deficiency is responsible for subsequent irregularities in these hormone systems. Abnormal activity of CCK may also be implicated here.

The second prong of the inventors' proposal is based on the involvement of TPST in sulphating the mucins of the gastrointestinal tract. Secretions of the gastrointestinal tract that contain glycoprotein mucins are viscous and sticky and are collectively termed mucus. Mucus adheres to the gastric mucosa and helps to protect the mucosal surface from abrasion by lumps of food and from chemical damage caused by acids and digestive enzymes. T?ST is responsible for sulphating the mucins of the gastrointestinal tract, either by sulphating tyrosine residues in the mucins or by sulphating carbohydrates. Mucins are often made up of monomers that aggregate to the final product; sulphation of tyrosine residues occurs at an early stage and appears to control the aggregation process. Lack of sulphation of the mucins of the gastrointestinal tract by a paucity of TPST is therefore proposed by the inventors to reduce the integrity of the gut and lead to inflammation and dysfunction, making the gut “leaky”. This in turn may lead to excessive absorption of inadequately or abnormally digested foods.

The foods thought to pose the biggest problem if incorrectly digested are gluten and casein. Some of the dietary peptides derived from foods containing gluten and casein may have opioid activity and are thought to cause disruption to biochemical and neuroregulatory processes. Normally, endogenously produced opioid peptides (enkephalins and endorphins) perform a neuroregulatory role within the central nervous system (CNS) and as highlighted above they may act in balance with CCK.

However, the effect of endogenous opioids would be intensified by the exogenous source of opioids from excessive absorption of inadequately digested foods (and may be further intensified by an abnormality of TPST, and therefore CCK, function) severely disrupting normal processes within the CNS. As a consequence of this, perception; cognition; emotions; mood; behavior and higher executive functions would all be affected, resulting in the many and diverse symptoms of autism. Gluten and casein sensitivity are now considered to be, at least in part, primary to the aetiology of autism and are genetically determined.

CCK and gastrin, which are known to be activated by sulphation by TPST are also digestive enzymes. The inventors propose that a paucity of TPST as well as making the gastrointestinal tract “leaky” causes a reduction in the levels of active digestive enzymes, thus compounding the inadequate digestion of dietary foods in the gut.

The inventors therefore propose that autism may be attributed to the inadequate digestion of dietary protein caused by a “leaky” gastrointestinal tract or by inadequate digestive enzymes; the central effects of the resulting exogenous opioid peptides; or inadequate neuropeptide activation or a combination of two or three of these factors. As TPST activity can be implicated at each of these factors, it may have a role in the underlying aetiology of a number of disease states, but in autism in particular and thus a reduction in TPST level in a patient sample as compared to a reference sample provides a suitable means for diagnosing or detecting a predisposition to autism and several diseases which involve at least one of factors above implicated in autism.

Diseases other than autism that are associated with a “leaky” gastrointestinal tract or by inadequate digestive enzymes leading to increased opioid activity which may be diagnosed or a predisposition to detected according to the method of the first aspect of the invention include, but are not limited to, disorders involving gluten or casein intolerance or food allergy, for example Attention Deficit disorder (with or without associated hyperactivity); chronic fatigue syndrome; childhood-onset obsessive compulsive disorder; Crohn's disease; Coeliac disease, particularly gluten-sensitive ataxics; dyspraxia; eating disorders such as anorexia and Irritable Bowel Syndrome; obsessive compulsive disorder; schizophrenia; schizoaffective disorder and Ulcerative colitis.

Diseases other than autism that are associated with reduced CCK activity which may be diagnosed or a predisposition to detected according to the method of the first aspect of the invention include, but are not limited to, Attention deficit hyperactivity disorder; Childhood-onset obsessive compulsive disorder; Chronic fatigue Syndrome; Diseases commonly treated with dopamine antagonists; Obsessive compulsive disorder; Prader Willi Syndrome; Schizophrenia; schizoaffective disorder and other diseases which have an associated abnormal CCK activity.

Other diseases which may be diagnosed or a predisposition to detected according to the method of the first aspect of the invention are those that are implicated as being genetically linked to autism, including, but not limited to, Angelman's syndrome, Rheumatic fever; Sydenham's Chorea; Tourette's Syndrome and the development of tics, as well as those diseases involving language problems and obsessive compulsive traits.

The bodily sample may be taken from bodily fluid or tissue samples, preferably from whole blood, plasma, or serum.

In a preferred embodiment the sample will be a platelet sample, preferably derived from whole blood.

The assay according to the first aspect of the invention may be for TPST protein to determine the concentration or activity thereof; for TPST DNA to determine the level of expression thereof or a polymorphism therein which reduces expression of TPST or encodes a TPST protein of reduced activity; or to determine a change in concentration or activity of a TPST modulator.

In its embodiment relating to detection of the concentration of protein in a sample the assay according to the first aspect of the invention may comprise methods including radioimmunoassay, Western Blot analysis, competitive-binding assays and ELISA.

Methods for the investigation of TPST protein activity generally involve transfer of radiolabelled sulphur from the sulphate donor, PAPS, to an unsulphated acceptor, which can be a natural or synthetic peptide containing a tyrosine residue. This is followed by separation by chromatography, using filter paper, Sep-Pak cartridges or polystyrene bead columns.

The radio-isotopic assay method of Foldes and Meek (1973) to assess phenolsulphotransferase activity was adapted for investigation of dehydroepiandrosterone sulphotransferase (DHEA-ST) activity by Aldred (1999). This method involves a simple incubation of reaction mixtures, followed by precipitation of unreacted radiolabelled sulphate by barium hydroxide and zinc sulphate. For the research into TPST activity described herein, this method has been adapted by removing the need for a separation step to provide a much improved, and quicker method of detecting TPST activity, giving clear reproducible results.

Suitable assay methods for TPST protein activity are described in Sane & Baker (1993) and Rens-Domiano & Roth (1989). An alternative method found to be easier and provide reliable results is provided as Example 1 below.

In its embodiment relating to the level of nucleic acid in the sample suitable methods include hybridisation, sequencing or amplification techniques.

Suitable primers for the detection of TPST nucleic acid are described in Beisswanger, R. et al., PNAS Vol. 95, Issue 19, 11134-11139 (1998) and provided below as:

Forward primer

SEQ ID NO. 1

5′ GGGAAAGCTTCCAGCATGCGCCTGTCGGTGCGGAGG 3′
Reverse primer

SEQ ID NO. 2

5′ GCTCTAGATTAAATGCATTTTCTTCTCTTCTTGG 3′

These two primers have been used to amplify the entire ORF of human TPST-2 cDNA by PCR, using cloned Pfu polymerase (Stratagene). After identification of the 5′ untranslated region/ORF and ORF/3′ untranslated region boundaries by analysis of the BAC 445C9 and expressed sequence tag (EST) sequences the forward and reverse primers were designed based on the BAC 445C9 sequence.

The forward primer corresponds to the sequence surrounding the translational start codon (underlined) and introduces a HindIII site (shown in italics). The reverse primer corresponds to a sequence about 30-60 nucleotides 3′ to the translational stop codon and introduces an XbaI site (shown in italics).

Other primers suitable for the detection and isolation of TSPT nucleic acid may be envisaged by those skilled in the art, since the full-length sequence of TPST is known.

The term “polymorphism” refers to a different gene sequence from the wild type. Polymorphisms can be variants that are generally found between individuals of different ethnic backgrounds or from different geographical areas, those polymorphisms not affecting the function of the gene. Other polymorphisms are those which lead to differences in the function of the gene or may produce an inactive gene product or may modulate the production of the gene product.

For detecting a polymorphism that causes a reduction expression of the TPST gene or encodes a TPST protein of reduced activity suitable methods include DNA sequencing, restriction fragment length study by electrophoresis, nuclease protection assays, such as RNase and S1 protection, chemical cleavage, hybridisation, single strand confirmation polymorphism analysis and heteroduplex analysis, HPLC analysis and Southern blotting.

The method according to the first aspect of the invention may also detect reduced TPST levels indirectly by assaying for reduced level of active CCK, which may be carried out using the same methods as described in relation to assaying for reduced TPST levels.

The above assays can be provided in kit form to diagnose or detect a predisposition to autism and related diseases.

As autism has been shown to be associated with a reduced level of TPST it follows that administration to a patient of means of increasing TPST could be useful for the treatment or prophylaxis of autism and diseases proposed to be related to autism.

Accordingly the second aspect of the invention provides a method of treatment or prophylaxis of autistic patients comprising administering TPST or a TPST enhancer.

According to the third and fourth aspects of the invention TPST protein or an enhancer thereof can be used for the manufacture of a medicament for use in the treatment or prophylaxis of autism or related diseases.

According to the fifth aspect of the invention the TPST gene or an enhancer of expression thereof can be used for the manufacture of a medicament for use in the treatment or prophylaxis of autism or related diseases.

According to the sixth aspect of the invention a CCK or an activator of CCK protein or gene expression can be used for the manufacture of a medicament for use in the treatment or prophylaxis of autism or related diseases.

Treatment of autism or related diseases with compounds according to the invention may be either as a monotherapy or in combination with other therapeutic agents.

The enhancer of TPST activity used according to the second aspect of the invention may take a number of different forms depending, in particular on the manner in which the composition is to be used. Thus, for example, the composition may be in the form of a powder, tablet, capsule, liquid, emulsion, ointment, cream, gel, hydrogel, aerosol, spray, micelle, liposome or any other suitable form that may be administered to a person or animal. It will be appreciated that the vehicle of the composition of the invention should be one which is well tolerated by the subject to whom it is given and enables delivery of the compounds to the site of action.

Compositions that are TPST enhancers may be used in a number of ways. For instance, systemic administration may be required in which case a suitable compound may be contained within a composition which may, for example, be ingested orally in the form of a tablet, capsule or liquid. Alternatively the composition may be administered by injection into the blood stream. Injections may be intravenous (bolus or infusion) or subcutaneous (bolus or infusion).

The TPST enhancer may also be incorporated within a slow or delayed release device. Such devices may, for example, be inserted under the skin and the compound which enhances TPST may be released over weeks or even months. The devices may be particularly advantageous when a compound is used which would normally require frequent administration (e.g. at least daily ingestion of a tablet or daily injection).

It will be appreciated that the amount of a compound required is determined by biological activity and bioavailability which in turn depends on the mode of administration, the physicochemical properties of the compound employed and whether the compound is being used as a monotherapy or in a combined therapy. The frequency of administration will also be influenced by the above mentioned factors and particularly the half-life of the compound within the subject being treated.

Known procedures, such as those conventionally employed by the pharmaceutical industry (e.g. in vivo experimentation, clinical trials etc), may be used to establish specific formulations of compositions and precise therapeutic regimes (such as daily doses of the compounds and the frequency of administration).

Generally, a daily dose of between 0.01 μg/kg of body weight and 1.0 g/kg of body weight of a compound which enhances TPST may be used depending upon which specific compound is used and the condition to be treated. More preferably the daily dose is between 0.01 mg/kg of body weight and 100 mg/kg of body weight.

Daily doses may be given as a single administration (e.g. a daily tablet for oral consumption or as a single daily injection). Alternatively the compound used may require administration twice or more times during a day. A patient receiving treatment may take a first dose upon waking and then a second dose in the evening (if on a two dose regime) or at 3 or 4 hourly intervals thereafter. Alternatively a slow release device may be used to provide optimal doses to a patient without the need to administer repeated doses.

According to a preferred embodiment the inventors propose the use of dietary components to enhance TPST activity for potential therapeutic benefit. Benefit can be derived from either a modulation of gut TPST to enhance mucin sulphation and thereby reduce the ‘leakiness’ of the gut; enhance digestion by promoting adequate formation of digestive enzymes such as CCK and gastrin or improvement in brain function (or avoidance of deterioration) through promotion of adequate sulphation of central neuropeptides crucial for normal brain development and/or function.

TPST enhancers which may be administered according to the second aspect of the invention include, but are not limited to ethanol, fatty acids such as oleic acid, linoleic acid, butyric acid, myristate, laurate, arachidonate, palmitate, eicosapentanoic acid (EPA), docosahexanoic acid (DHA) and gamma linolenic acid (GLA), the amino acids aspartate, glutamine and citrate, PGE₂, 6-keto-PGF_{1α} and PGF_{2α} and those agents which activate CCK.

Other TPST enhancers which may be administered according to the second aspect of the invention include, but are not limited to particular flavonoids, such as flavone, 5-OH flavone and daidzein or their dietary precursors. The inventors have found that the activity of the flavonoid in enhancing TPST activity decreases as the number of hydroxyl groups on the flavonoid increases. Whilst not wishing to be bound by theory the inventors propose that the active site of TSPT is arranged such that the more hydroxyl groups on a compound (i.e. increased size of compound) the less likely the compound will be able to reach the active site of TPST to have its enhancing effect. This will have an impact in the design of new TPST enhancers, which should include no, or only very few hydroxyl groups.

The present invention will now be described, by way of example only, with reference to the following drawings, in which

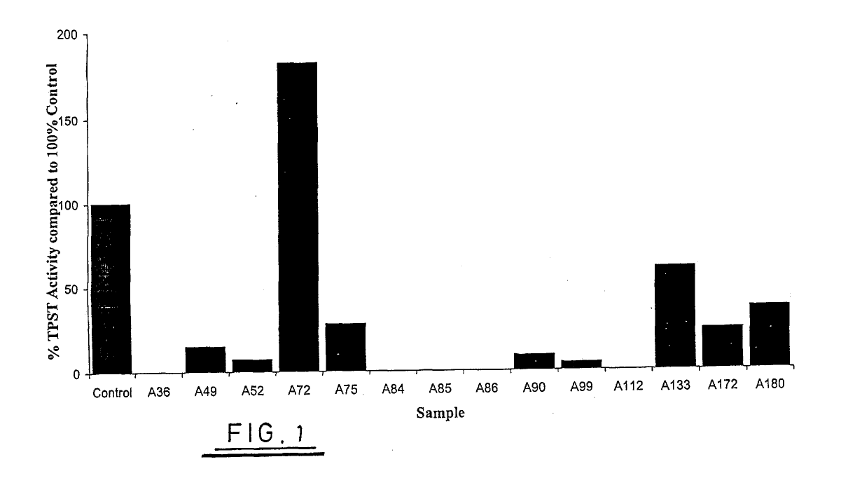

FIG. 1 shows the TPST activity in platelets from 14 autistic children compared to a pooled (n=5) control platelet sample;

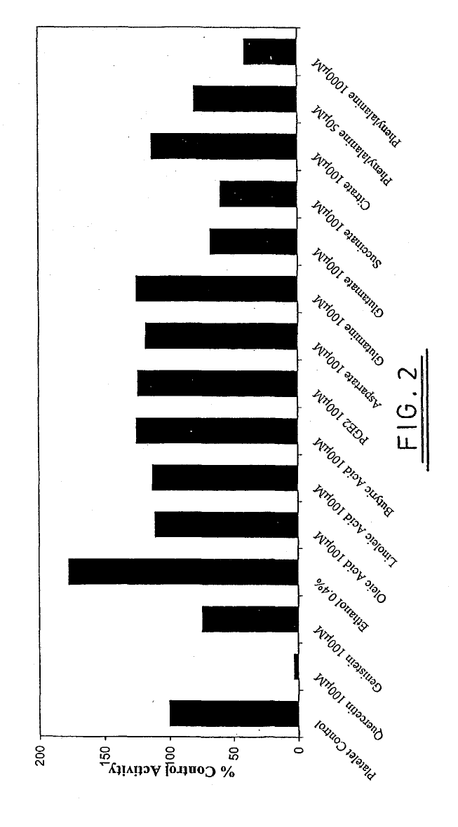

FIG. 2 shows the effect of various compounds on T?ST activity from normal human platelets and demonstrates that activity can be either stimulated or inhibited depending on the compound of choice;

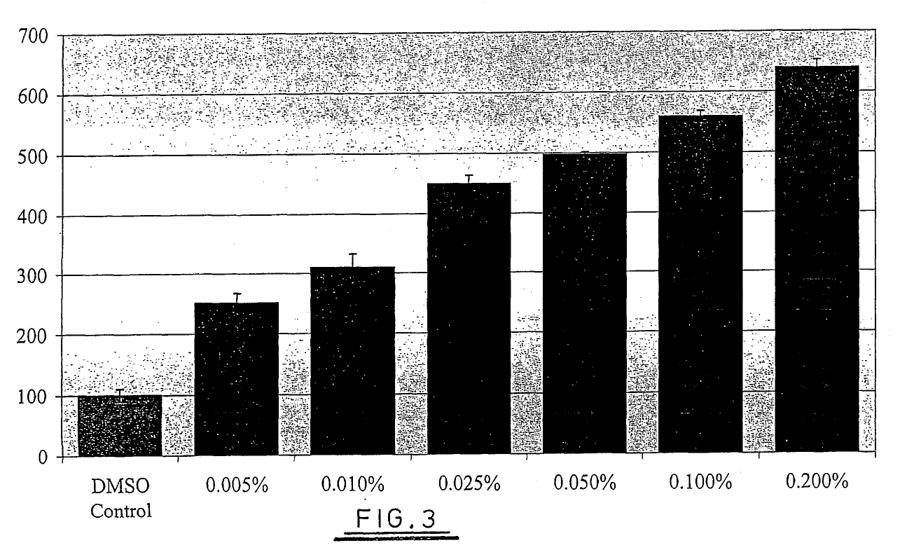

FIG. 3 shows the effect of soya extract on TPST activity;

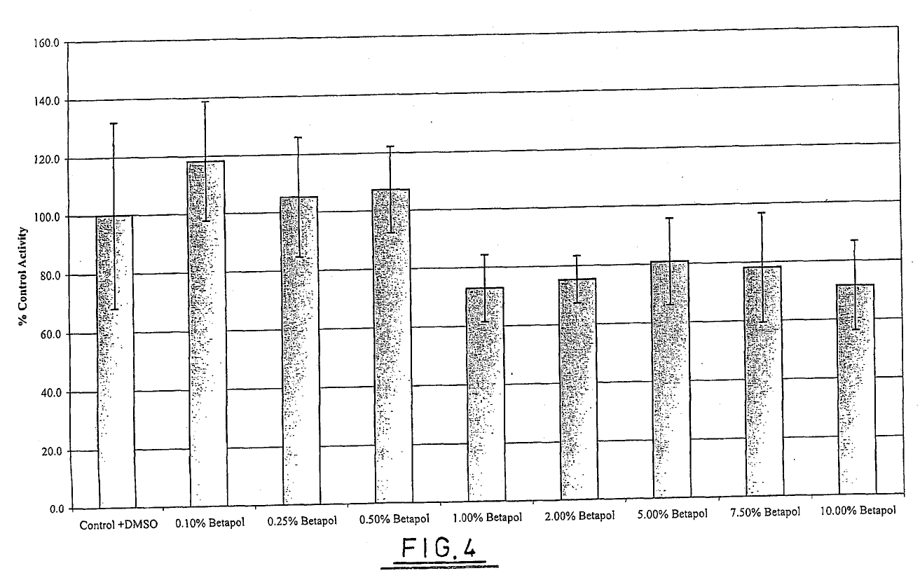

FIG. 4 shows the effect of betapol on TPST activity;

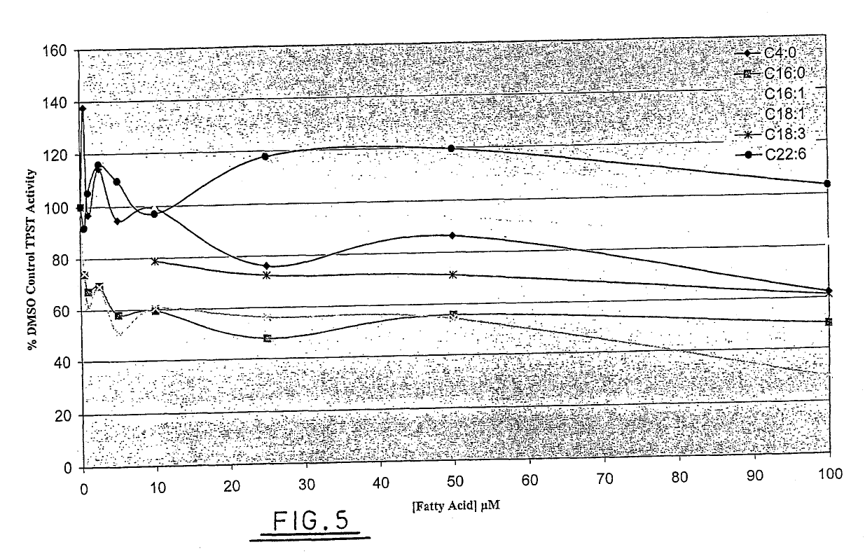

FIG. 5 shows the effect of fatty acids on TPST activity;

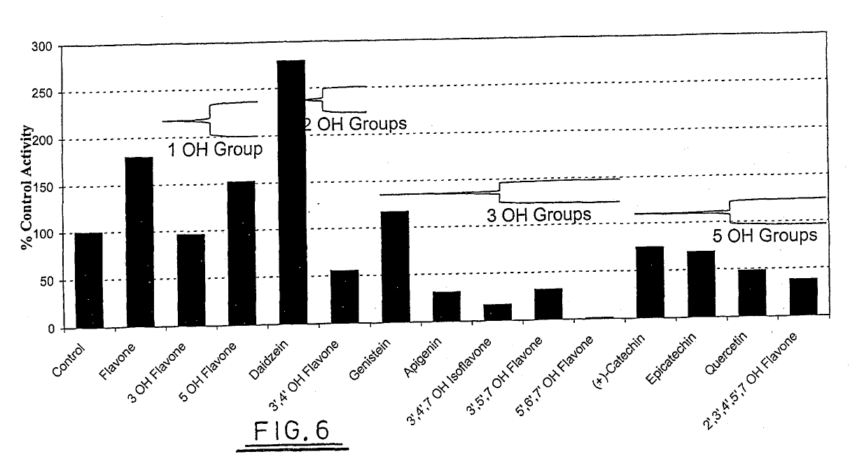

FIG. 6 shows the effects of various flavonoids on TPST activity;

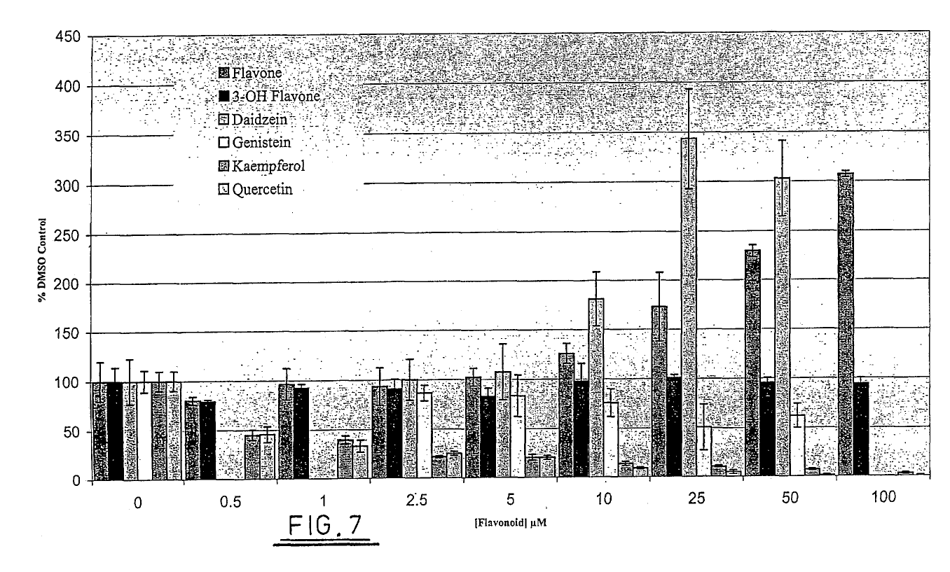

FIG. 7 shows the effects of various flavonoids on TPST activity;

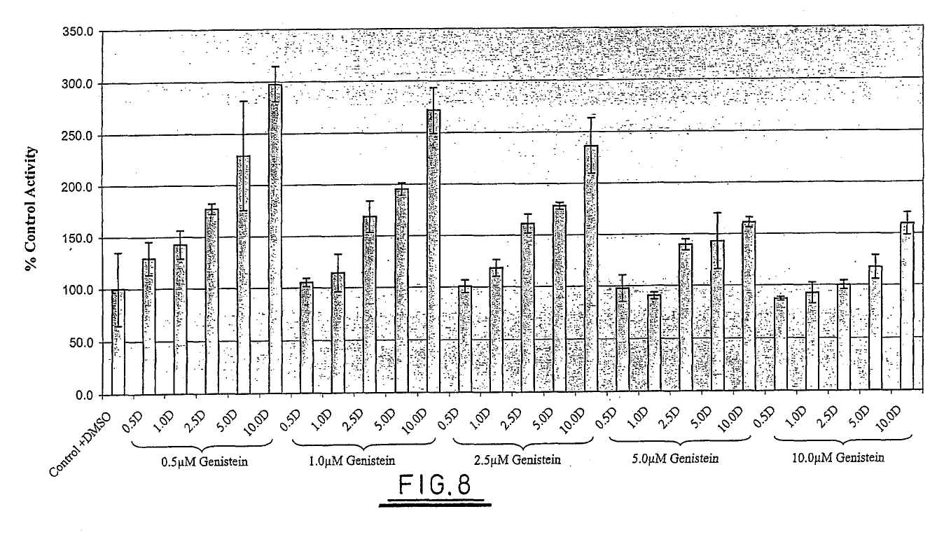

FIG. 8 shows the effects of varying concentrations of daidzein and genestein on TPST activity; and

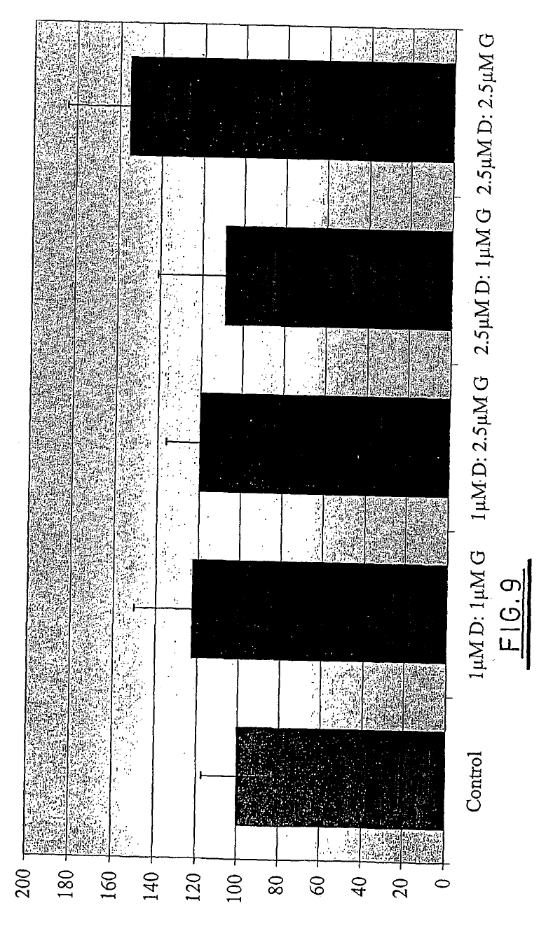

FIG. 9 shows the effects of varying mixes of daidzein and genestein on TPST activity.

### EXAMPLES

1. Assay for TPST

The activity of sulphotransferases can be measured by monitoring the transfer of ³⁵S from the sulphate donor, PAPS³⁵, to the product, in this case the synthetic tyrosine substrate EAY. This assay uses platelets from control subjects (n=5) and those patients suffering from a particular disorder, selected from autism (n=14), irritable bowel syndrome (n=3) schizophrenia (gluten-sensitive)(n=4), food allergy (gluten, milk) (n=3) and depression (n=2).

Reaction mixtures were incubated for 50 minutes at 37° C. and reactions were stopped by barium acetate. Unreacted sulphate, proteins and nucleotides were precipitated by zinc sulphate and barium hydroxide. Within the reaction there is a positive and negative control. The positive control is termed 100%, and represents the total amount of radioactivity present in the hot PAPS³⁵ sample. The negative control is the “no-enzyme” reaction, this represents the amount of sulphate transferred to the product when no enzyme is present, i.e. the chemical reaction. The blank for the reaction is made up without PAPS³⁵ and without enzyme.

Materials & Methods

Reaction mixtures were set up as shown in the table below.

| | Buffer | PAPS³⁵ | Substrate | Homogenate |
|---|---|---|---|---|
| | (μl) | (μl) | (μl) | (μl) |
| 100% | 130 | 20 | — | — |
| Blank | 150 | — | — | — |
| No enzyme | 120 | 20 | 10 | — |
| Test | 100 | 20 | 10 | 20 |

Platelet Sample

10 ml of whole blood was taken from patients and the blood cells removed. The remaining plasma was centrifuged at 8000 g for 5 mins to sediment the platelets. This platelet pellet was resuspended in 5 ml 10 mM phosphate buffered saline (PBS) containing 10 mM EDTA, recentrifuged at 2000 g at 4° C. for 30 min. This step was repeated and the remaining pellet resuspended in 5 ml 20% glycerol, 10 mM MES, 1 mM DTT (adapted from Sane and Baker, 1993). The platelets were sonicated for 12×10 second bursts at 4° C. The platelet sonicates were stored in 1 ml portions at −70° C. prior to use.

| | | | |
|---|---|---|---|
| Buffer: | MES pH 6.5 | 25 | mM |
| | β-mercaptoethanol | 5 | mM |
| | MgCl2 | 5 | mM |
| | MnCl2 | 5 | mM |
| | NaF | 20 | mM |
| | CHAPS | 1% | |

Substrate: 10 μl EAY (300 μM; poly (Glu, Ala, Tyr) 6:3:1) was used in each test assay, to give a final concentration in the reaction of 20 μM. This solution was made up in MES reaction buffer.

Reaction mixtures were made up as in the table above. To begin the assay, platelet homogenate (20 μl) and PAPS³⁵ (20 μl) were added at 30-second intervals to a final volume of 150 μl. The reaction mixture was then incubated at 37° C. for 40 min. The reaction is stopped by the addition of 200 μl-barium acetate (0.1 M) and the tubes were vortexed and placed on ice for 5 ml.

Unreacted sulphate was then precipitated by the addition of 0.1 M zinc sulphate (200 μl) and 0.1 M barium hydroxide (200 μl). The tubes were vortexed and centrifuged at 6500 rpm for 3 min.

Repeat step 3. Supernatant (500 μl) was then added to scintillation fluid (3.5 ml) and S³⁵ was counted in a Phillips scintillation counter.

Results

Preliminary experiments investigating the differences between control platelets and autistic children samples were performed. Frozen samples of platelets from a number of autistic children were collected and stored at −20° C. until assay. Platelets from normal healthy blood donors collected at approximately the same time and stored and handled in the same way were used as controls to compensate for any decreases in TPST activity occurring throughout storage. These samples were defrosted and 1 ml samples taken and prepared for the TPST precipitation reactions.

Initial results indicate that autistic samples have decreased activity compared to control platelet samples. Table 1 and FIG. 1 show the results of this study. These experiments indicate that there is decreased TPST activity in the autistic children's samples compared to control platelets.

TABLE 1

| | % TPST Activity |
|---|---|
| Autistic Sample | compared to Control (100%) |
| A36 | 0 |
| A49 | 14.7 |
| A52 | 7.03 |
| A72 | 182 |
| A75 | 27.4 |
| A84 | 0 |
| A85 | 0 |
| A86 | 0 |
| A90 | 8.6 |
| A99 | 4.05 |
| A112 | 0 |
| A133 | 60.1 |
| A172 | 23.7 |
| A180 | 36.1 |

Results for diseases other than autism are provided below as percentage decrease in TPST activity as compared to control activity:

| | |
|---|---|
| Irritable bowel syndrome | 38% |
| Schizophrenia (gluten-sensitive) | 58% |
| Food allergy (gluten, milk) | 62% |
| Depression | 28% |

The results appear to show that patients suffering from autism, irritable bowel syndrome, schizophrenia (gluten-sensitive), food allergy (gluten, milk) and depression all exhibit lower than expected TPST activity.

2: Effect of Various Dietary Factors on TPST Activity

The effect of various compounds on TPST activity was investigated in platelets from normal healthy blood donors using the assay described in Example 1. Various compounds were investigated, including fatty acids, flavonoids and amino acids. The results can be seen in the Table 2, 3, 4 and 5 below and in FIGS. 2, 3, 4 and 5. Compounds were tested at the concentrations shown and were dissolved in buffer where possible or ethanol if not.

NOVASOY™ is a soya-based dietary supplement ingredient rich in genistein and daidzin available from Archer Daniels Midland Nutraceuticals of 4666 E. Faries Parkway, Decatur, Ill. 62526 USA. It is carefully extracted to ensure that the ratios of various isoflavones are available as they would be in soya beans themselves and in natural soya foods such as tofu.

BETAPOL™ is a human milk fat (HMF) replacer for infant food manufactured by Loders Croklaan, Inc. Lipid Nutrition of 24708 W. Durkee Road, Channahon, Ill. 60410-5249 USA. BETAPOL™ offers a vegtable fat blend that closely mimics the physical and chemical structure of HF.

TABLE 2

| | % Control TPST Activity |
|---|---|
| Platelet Control | 100 |
| Quercetin 100 μM | 3 |
| Genistein 100 μM | 74 |
| Ethanol 0.4% | 177 |
| Oleic Acid 100 μM | 111 |
| Linoleic Acid 100 μM | 113 |
| Butyric Acid 100 μM | 125 |
| PGE2 100 μM | 124 |
| Aspartate 100 μM | 118 |
| Glutamine 100 μM | 125 |
| Glutamate 100 μM | 67 |
| Succinate 100 μM | 59 |
| Citrate 100 μM | 113 |
| Phenylalanine 50 μM | 79 |
| Phenylalanine 1000 μM | 40 |

TABLE 3

| [NOVASOY ™] % | % Control Activity | % Std Deviation |
|---|---|---|
| 0.000 | 100.0 | 11.0 |
| 0.005 | 251.2 | 17.0 |
| 0.010 | 309.3 | 23.0 |
| 0.025 | 450.5 | 14.0 |
| 0.050 | 497.7 | 4.0 |
| 0.100 | 559.0 | 9.0 |
| 0.200 | 639.4 | 13.0 |

TABLE 4

| [BETSPOL ™] % | Control Activity | % Std Deviation |
|---|---|---|
| 0.00 | 100.0 | 31.9 |
| 0.10 | 118.2 | 20.6 |
| 0.25 | 105.4 | 20.6 |
| 0.50 | 107.3 | 14.9 |
| 1.00 | 72.8 | 11.4 |
| 2.00 | 75.3 | 8.1 |
| 5.00 | 80.8 | 14.9 |
| 7.50 | 78.1 | 18.7 |
| 10.00 | 71.5 | 15.2 |

TABLE 5

| | C4:0 | C16:0 | C16:1 | C18:1 | C18:3 | C22:6 |
|---|---|---|---|---|---|---|
| [Fatty Acid] μM | Butyrate | Palmitate | Palmitoleic | Oleic | GLA | DHA |
| 0 | 100.0 | 100.0 | 100.0 | 100.0 | 100.0 | 100.0 |
| 0.5 | 137.6 | 74.2 | | 74.5 | | 91.8 |
| 1 | 96.8 | 67.4 | | 61.5 | | 105.2 |
| 2.5 | 114.3 | 69.6 | | 69.2 | | 115.9 |
| 5 | 94.6 | 58.1 | | 51.4 | | 109.6 |
| 10 | 99.2 | 59.6 | 99.5 | 60.5 | 78.8 | 97.0 |
| 25 | 76.1 | 48.0 | 95.2 | 56.2 | 72.6 | 117.8 |
| 50 | 86.4 | 55.7 | 93.5 | 54.3 | 71.3 | 119.4 |
| 100 | 62.2 | 50.0 | 81.4 | 29.5 | 61.2 | 103.3 |
| Std Deviation | | | | | | |
| 0 | 16.3 | 16.6 | 22.0 | 15.3 | 11.0 | 20.0 |
| 0.5 | 26.0 | 4.1 | | 11.8 | | 14.0 |
| 1 | 10.2 | 19.6 | | 4.9 | | 17.0 |
| 2.5 | 9.7 | 15.7 | | 14.9 | | 17.0 |
| 5 | 37.2 | 6.5 | | 3.6 | | 24.0 |
| 10 | 13.2 | 9.9 | 4.0 | 8.3 | 5.0 | 15.0 |
| 25 | 2.8 | 10.9 | 4.0 | 5.1 | 11.0 | 22.0 |
| 50 | 9.6 | 13.7 | 15.0 | 5.7 | 12.0 | 13.0 |
| 100 | 9.4 | 10.2 | 6.0 | 6.0 | 4.0 | 13.0 |

The results show that dietary factors have differing effects on TPST activity. Several of the results are in agreement with previously published results using other methods of TPST analysis. The slight stimulation of TPST activity be oleic acid is in agreement with work carried out by Kasinathan et al., (1993); the stimulation of activity by ethanol and prostaglandin E₂ is in agreement with work performed by Kasinathan et al., (1995) in rat submandibular salivary glands.

Amino acids were seen to have different effects of TPST activity. Two acidic amino acids, aspartate and glutamate, were investigated and surprisingly these substances were seen to have opposite effects on enzyme activity. Aspartate increased activity, whilst glutamate lead to decreased activity. In naturally occurring substrates for TPST, the sulphated tyrosine residues are predominantly surrounded by acidic amino acids and increased tyrosine sulphation of substrates containing acidic amino acids by TPST has been reported. This suggests that acidic environments are the important determinants for tyrosine sulphation. The amide derivative of glutamate, glutamine, however, resulted in increased activity. The citric acid cycle intermediates, succinate and citrate, had opposite effects on TPST activity with succinate decreasing sulphation and citrate increasing activity. Succinate inhibited TPST activity to a similar degree to glutamate. Both these compounds have the same number of negative charges and therefore this could contribute to the result. The aromatic amino acid phenylalanine decreased TPST activity in a dose dependent fashion, with increased inhibition occurring at higher concentrations. This substance may have acted by competitively inhibiting TPST as it has a similar structure to tyrosine.

3: Effect of Flavonoids on TPST Activity in Platelets

The effects of various flavonoids on TPST activity have been studied in human platelets using the assay described in Example 1. The results can be seen in Tables 6, 7, 8 and 9 and FIGS. 6, 7, 8 and 9.

In the Example shown in Table 6 and FIG. 6 compounds were all dissolved in DMSO and tested at 25 μM. Genistein, flavone and daidzein at 25 μM concentration resulted in stimulation of TPST activity in platelets. Increased TPST inhibition was seen to occur as the number of hydroxyl groups present on the compound increased.

The results of Examples 2 and 3 illustrates a concentration dependent effect of genistein, which in Example 2, at a concentration of 100 μM was shown to have an inhibitory effect on TPST activity and in Example 3, at a concentration of 25 μM was shown to have a TPST enhancing effect. The concentration effect of genistein is well documented and may also be present for other compounds. Therefore compounds shown to have a TPST inhibitory effect at 25 μM may be found to enhance TPST activity when present at a different concentration.

TABLE 6

| Flavonoid | No. of OH Groups | % Control TPST Activity |
|---|---|---|
| Control | — | 100 |
| Flavone | 0 | 180 |
| 3 OH Flavone | 1 | 96 |
| 5 OH Flavone | 1 | 152 |
| Daidzein | 2 | 279 |
| 3′,4′ OH Flavone | 2 | 55 |
| Genistein | 3 | 117 |
| Apigenin | 3 | 31 |
| 3′,4′,7 OH Isoflavone | 3 | 17 |
| 3′,5′,7 OH Flavone | 3 | 32 |
| 5′,6′,7′ OH Flavone | 3 | 1 |
| (+)-Catechin | 5 | 74 |
| Epicatechin | 5 | 68 |
| Quercetin | 5 | 48 |
| 2′,3′,4′,5′,7 OH Flavone | 5 | 38 |

TABLE 7

| | Flavone | 3-OH Flavone | Daidzein | Genistein | Kaempferol | Quercetin |
|---|---|---|---|---|---|---|
| [Flavonoid] μM | 0 | 1 | 2 | 3 | 4 | 5 |
| 0 | 100 | 100 | 100 | 100 | 100 | 100 |
| 0.5 | 80.3 | 79 | | | 45.2 | 45.8 |
| 1 | 96.4 | 92.6 | | | 39.9 | 33.7 |
| 2.5 | 93.5 | 90.7 | 100.2 | 86.5 | 22.3 | 25.7 |
| 5 | 102.3 | 83 | 107.5 | 83.4 | 21.4 | 21.1 |
| 10 | 126 | 97.9 | 181.1 | 75.8 | 14.4 | 9.5 |
| 25 | 172.8 | 101.1 | 343.2 | 50.9 | 11 | 5 |
| 50 | 230.2 | 95.5 | 303.1 | 62 | 7.3 | 0.9 |
| 100 | 307.3 | 94.4 | | | 2.9 | 0 |
| Std Deviation | | | | | | |
| 0 | 20 | 14 | 23.03 | 11 | 10 | 10 |
| 0.5 | 4 | 2 | | | 5 | 8 |
| 1 | 16 | 4 | | | 4 | 6 |
| 2.5 | 19 | 10 | 20.85 | 8 | 1 | 2 |
| 5 | 9 | 8 | 28.34 | 21 | 3 | 2 |
| 10 | 10 | 18 | 27.54 | 14 | 2 | 1 |
| 25 | 35 | 3 | 103.09 | 23 | 1 | 2 |
| 50 | 6 | 5 | 37.64 | 12 | 1 | 1 |
| 100 | 3 | 6 | | | 1 | 1 |

TABLE 8

| | Treatment | % Activity of Control | Std Dev |
|---|---|---|---|
| 0.5 μM Genistein | Control + DMSO | 100.0 | 35 |
| | 0.5 D | 129.2 | 16 |
| | 1.0 D | 142.5 | 14 |
| | 2.5 D | 177.0 | 5 |
| | 5.0 D | 228.4 | 53 |
| | 10.0 D | 297.5 | 17 |
| 1.0 μM Genistein | 0.5 D | 105.3 | 4 |
| | 1.0 D | 114.2 | 18 |
| | 2.5 D | 168.7 | 15 |
| | 5.0 D | 195.2 | 6 |
| | 10.0 D | 271.2 | 22 |
| 2.5 μM Genistein | 0.5 D | 100.7 | 6 |
| | 1.0 D | 118.1 | 8 |
| | 2.5 D | 161.2 | 9 |
| | 5.0 D | 178.0 | 3.3 |
| | 10.0 D | 236.1 | 26.8 |
| 5.0 μM Genistein | 0.5 D | 97.7 | 12.8 |
| | 1.0 D | 90.6 | 3.3 |
| | 2.5 D | 139.5 | 5.4 |
| | 5.0 D | 142.9 | 27.0 |
| | 10.0 D | 161.0 | 4.6 |
| 10.0 μM Genistein | 0.5 D | 86.9 | 1.8 |
| | 1.0 D | 92.4 | 10.0 |
| | 2.5 D | 100.2 | 4.2 |
| | 5.0 D | 117.0 | 11.5 |
| | 10.0 D | 159.3 | 10.8 |

TABLE 9

| | % Activity | % Std Dev |
|---|---|---|
| Control | 100.0 | 17.2 |
| 1 μM D: 1 μM G | 122.5 | 27.6 |
| 1 μM D: 2.5 μM G | 120.0 | 16.1 |
| 2.5 μM D: 1 μM G | 108.8 | 32.2 |
| 2.5 μM D: 2.5 μM G | 155.1 | 29.0 |

### REFERENCES

Aldred S & Waring R H: Localisation of dehydroepiandrosterone in adult rat brain. Brain Res. Bulletin 1999;48(3) 291-296.

Foldes, A & Meek J L: Rat brain phenolsulphotransferase—partial purification & some properties. Biochem Biophys Acta 1973;327: 365-374.

Kasinathan C et al. Effect of prostaglandins on tyrosylprotein sulfotransferase activity in rat submandibular glands. Gen Pharmac 1995;26(3):577-580.

Kasinathan C et al. Inhibition of tyrosyl protein sulfotransferase by sphingosine and its reversal by acidic phospholipids. Biochemistry 1993;32:1194-1198.

O'Reilly B A & Waring R H. Enzyme and sulphur oxidation deficiencies in autistic children with known food/chemical intolerances. J Orthomolec Med 1993;8(4):198-200.

Ouyang Y-B et al. Tyrosyl protein sulfotransferase:Purification and molecular cloning of an enzyme that catalyses tyrosine O-sulfation, a common posttranslational modification of eukaryotic proteins.

Panksepp J. A neurochemical theory of autism. TINS 1979;2:174-177.

Sane D C & Baker M S: Human Platelets possess tyrosylproteinsulphotransferase activity. Thromb Haem 1993;69:272-275.

Waring R H et al. Biochemical parameters in autistic children. Dev Brain Dysfunct 1997;10:40-43.

1. A method for diagnosing or detecting a predisposition to autism and related diseases comprising assaying a bodily sample in vitro directly or indirectly for a reduced tyrosyl protein sulphotransferase level as compared to a reference sample.

2. A method according to claim 1 in which the bodily sample comprises whole blood, plasma, or serum.

3. A method according to claim 1 in which TPST level is determined by TPST protein activity.

4. A method according to claim 3 in which TPST protein activity is determined by transfer of radiolabelled sulphur from a sulphur donor to an unsulphated tyrosine acceptor.

5. A method according to claim 1 in which TPST levels is determined by TPST protein concentration.

6. A method according to claim 5 in which TPST protein concentration is determined by radioimmunoassay, Western Blot analysis, competitive-binding assays or ELISA.

7. A method according to claim 1 in which the TPST level is determined by detecting TPST nucleic acid.

8. A method according to claim 7 in which TPST nucleic acid is determined by hybridisation, sequencing or amplification techniques.

9. A method according to claim 7 comprising assaying for a polymorphism in the TPST gene which reduces the expression or activity of TPST protein.

10. A method according to claim 9 in which assaying for a polymorphism is carried out by DNA sequencing, restriction fragment length study, electrophoresis, nuclease protection assays including RNase and S1 protection, chemical cleavage, hybridisation, single strand confirmation polymorphism analysis and heteroduplex analysis, HPLC analysis or Southern blotting.

11. A method according to claim 1 in which TPST level is determined indirectly by assaying for a reduced level of active cholecystokinin.

12. A kit for diagnosing or detecting a predisposition to autism and related diseases comprising vessels and reagents suitable for assaying the level of TPST in a bodily sample and comparing the level to the level of TPST in a reference sample.

13. A method of treatment or prophylaxis for individuals with autism or related diseases or having a predisposition thereto comprising administrating to a patient means of increasing TPST levels.

14. A method according to claim 13 comprising administering to autistic patients TPST or a TPST enhancer.

15. A method according to any preceding claim in which the diseases related to autism are those that are associated with a “leaky” gastrointestinal tract or by inadequate digestive enzymes leading to increased opioid activity, including disorders involving gluten or casein intolerance or food allergy; attention deficit disorder (with or without associated hyperactivity); childhood-onset obsessive compulsive disorder; Crohn's disease; Coeliac disease, particularly gluten-sensitive ataxics; dyspraxia; eating disorders such as anorexia; Irritable Bowel Syndrome; schizophrenia; schizoaffective disorder; obsessive compulsive disorder and Ulcerative colitis.

16. A method according to any one of claims 1 to 14 in which the diseases related to autism are those that are associated with reduced CCK activity, including Attention deficit hyperactivity disorder; Childhood-onset obsessive compulsive disorder; Chronic fatigue Syndrome; Diseases commonly treated with dopamine antagonists; Obsessive compulsive disorder; Prader Willi Syndrome; Schizophrenia; schizoaffective disorder and other diseases which have an associated abnormal cholecystokinin activity.

17. A method according to any one of claims 1 to 14 in which the diseases related to autism are those that are implicated as being genetically linked to autism, including Angelman's syndrome, Rheumatic fever; Sydenham's Chorea; Tourette's Syndrome and the development of tics, as well as those diseases involving language problems and obsessive compulsive traits.

18. Use of TPST for the manufacture of a medicament for use in the treatment or prophylaxis of autism or related diseases.

19. Use of an enhancer of TPST for the manufacture of a medicament for use in the treatment or prophylaxis of autism or related diseases.

20. Use of an enhancer of CCK for the manufacture of a medicament for use in the treatment or prophylaxis of autism or related diseases.

21. Use according to claim 19 or claim 20 in which enhancers of TPST or CCK include ethanol, fatty acids including oleic acid, linoleic acid, butyric acid, myristate, laurate, arachidonate, palmitate, eicosapentanoic acid (EPA), docosahexanoic acid (DHA) and gamma linolenic acid (GLA), flavonoids flavone, 5-OH flavone and daidzein, amino acids aspartate, glutamine and citrate, PGE₂, 6-keto-PGF_{1α} and PGF_{2α} and those agents which activate CCK.
